(19) United States
(12) Patent Application Publication
(10) Pub. No.: US 2012/0041555 A1
(43) Pub. Date: Feb. 16, 2012

(54) SILICONE IMPLANT WITH IMPRINTED TEXTURE

(75) Inventors: Nicholas J. Manesis, Summerland, CA (US); Futian Liu, Sunnyvale, CA (US)

(73) Assignee: ALLERGAN, INC., Irvine, CA (US)

(21) Appl. No.: 13/245,518

(22) Filed: Sep. 26, 2011

Related U.S. Application Data
(63) Continuation-in-part of application No. 13/104,820, filed on May 10, 2011.
(60) Provisional application No. 61/333,146, filed on May 10, 2010.
(60) Provisional application No. 61/387,381, filed on Sep. 28, 2010.

(51) Int. Cl. A61F 2/12; B32B 5/18; B05D 3/10

(52) U.S. Cl. 623/8; 427/2.24; 156/77

(57) ABSTRACT

A procedure for making an implant having a textured silicone surface is disclosed. The method may include the steps of providing a conventional mandrel and applying a pre-formed, polymeric mesh sock to the mandrel. The sock is contacted with a silicone dispersion and the silicone dispersion is at least partially cured with the sock in contact therewith. The silicone dispersion is at least partially cured while the sock is in contact therewith and the sock is them removed, for example, by dissolution, to leave a textured elastomeric material useful as a component of a breast implant shell.

### RELATED APPLICATION

This application is a continuation-in-part of U.S. patent application Ser. No. 13/104,820, filed on May 10, 2011 which claims priority to U.S. Provisional Patent Application No. 61/333,146 filed on May 10, 2010, and this application also claims priority to U.S. Provisional Patent Application No. 61/387,381, filed Sep. 28, 2010, each of these documents being incorporated herein in its entirety by this specific reference.

### BACKGROUND

Many medical applications involve the permanent or semi-permanent implantation of objects within the body of patients, for example breast implants, pacemakers, artificial joints, etc. Typically, these implantable devices are introduced into a patient's body during a surgical procedure, which involves the creation of an incision, insertion of the implant, and closing of the open wound. The patient may then heal around the implant which may be permanently incorporated into the person's body.

A natural part of that healing process includes the formation of a capsule surrounding the implant. The capsule is formed when fibroblasts, fibrous cells, grow around the surface of the implant, forming a tissue layer similar to scar tissue. Over time, the body naturally shrinks this capsule tissue. Although part of the healing process, complications may arise when the capsule tissue shrinks, known as capsule contracture. In particular, the capsule tissue may tighten around the implant deforming it, and possibly causing patient pain or discomfort.

Such complications are particularly problematic in the case of breast implants. Such implants, which may be introduced into a patient's body during cosmetic or reconstructive surgery, are typically constructed of silicone (a flexible material) and are designed to provide a natural shape, appearance, and feel. Capsule contracture may significantly alter the properties of the implant, however, compressing the implant and altering its appearance, and possibly causing significant discomfort. Accordingly, some example embodiments provide improved implants and processes for making implants which may reduce the severity and likelihood of complications arising from capsule contracture.

### SUMMARY

Some embodiments described herein provide procedures for making an implant having a textured silicone surface. Such example procedures may include forming a component having a silicone surface; pressing a plurality of polymer fibers at least partially into the silicone surface, before the silicone has completely cured; allowing the silicone to at least partially cure with the polymer fibers in the silicone surface; and after the silicone is at least partially cured, removing the polymer fibers from the silicone surface.

Some example procedures may also include forming a mesh from the polymer fibers before pressing the polymer fibers at least partially into the silicone surface. In some example embodiments, forming the mesh may further include weaving the polymer fibers in a repeating pattern. In some example embodiments, the mesh may be made of a plurality of layers. And in some example procedures the mesh may include a plurality of eyes having an average size between about 100 μm×100 μm to about 2000 μm×2000 μm.

Some example procedures may also include forming a felt from the polymer fibers before pressing the polymer fibers at least partially into the silicone surface.

In other example procedures, the polymer fibers may be made of at least one of Vicryl 910, poly (L-lactic acid-co-trimethylcarbonate), polycaprolactone, poly(methyl methacrylate), poly(L-lactic acid), poly(lactic-co-glycolic acid) or combinations thereof.

In some example procedures, removing the polymer fibers may include dissolving the polymer fibers in a solvent. The polymer fibers may not be soluble in at least one of xylene and toluene; but the polymer fibers may be soluble in at least one other organic solvent. And in some example procedures, the organic solvent may be one of methylene chloride, chloroform, acetone, and tetrahydrofuran.

In some example procedures removing the polymer fibers may further include removing the polymer fibers by hydrolytic degradation. The polymer fibers themselves may have an average thickness between about 100 μm and about 1000 μm.

Some example embodiments provide silicone medical devices produced according to any of the procedures disclosed in the present application. Such medical devices may be breast implants.

Further, described herein are breast implants, which may include a silicone shell having an inner surface, defining a cavity configured to be filled with a filler material, and an outer surface, the outer surface having a texture comprised of a plurality of protrusions and a plurality of interconnected recessed areas.

In some example breast implants the texture may be an imprint of a plurality of polymer fibers pressed into the outer surface of the silicone shell before the silicone shell is completely cured and removed after the silicone shell has at least partially cured. In other example embodiments the imprint may be of a mesh woven from the polymer fibers. In some example embodiments the imprint may be of a felt formed from the polymer fibers.

In some implants, the average vertical distance between a high point of a protrusion, in the plurality of protrusions, and a low point of recessed area, in the plurality of recessed areas, may be between about 100 μm and about 1000 μm.

In some example implants, the texture may include a plurality of tunnels.

Other example embodiments may provide a medical devices, which may include a silicone outer surface having a plurality of projections and a plurality of interconnected recessed areas, the average vertical distance between a high point of a protrusion, in the plurality of protrusions, and a low point of a recessed area, in the plurality of recessed areas, being between about 100 μm and about 1000 μm.

In some example medical devices the recessed areas may include channels with an average diameter between about 100 μm and about 1000 μm.

In some example medical devices at least some of the recessed areas may be smooth. In others at least some of the recessed areas may be textured.

In one aspect of the invention, methods of making a textured component of a breast implant shell are provided. The methods generally comprise the steps of

providing a mandrel, applying a polymeric mesh sock to the mandrel and contacting the polymeric mesh sock with a silicone dispersion. The methods further generally comprise the steps of at least partially curing the silicone dispersion with the sock in contact therewith to form a silicone elastomer, and, after the silicone is at least partially cured, removing the sock from the silicone elastomer to form a textured component of a breast implant shell. One or more additional polymeric mesh socks may be applied to the mandrel to create other, different textures.

The step of contacting the sock with the silicone dispersion may be performed after the step of applying the sock to the mandrel. In other embodiments, the step of contacting the sock with the silicone dispersion is performed before the step of applying the sock to the mandrel.

The polymeric mesh sock may be comprised of any suitable material that can be dissolved or otherwise removed from the cured silicone elastomer without substantially effecting the structure of the silicone elastomer. In some embodiments, the sock comprises a mesh made of at least one of Vicryl 910, poly (L-lactic acid-co-trimethylcarbonate), polycaprolactone, poly(L-lactic acid), poly(methyl methacrylate) and poly(lactic-co-glycolic acid). In some embodiments, the polymeric mesh sock comprises a mesh including a plurality of eyes having an average size between about 100 μm×100 μm to about 2000 μm×2000 μm.

The step of removing the sock from the silicone elastomer comprises contacting the sock with an organic solvent selected from methylene chloride, chloroform, acetone, tetrahydrofuran and combinations thereof.

The present invention further provides breast implant shells or components thereof, made by the processes and methods described herein.

### BRIEF DESCRIPTION OF THE DRAWINGS

The present disclosure will be more readily understood from a detailed description of example embodiments taken in conjunction with the following figures:

FIG. 1 illustrates a procedure, in accordance with an example embodiment.

FIG. 2 illustrates an example polymer mesh in accordance with an example embodiment.

FIG. 3 illustrates an example breast implant in accordance with an example embodiment.

FIG. 4 illustrates a close-up, top-down view of an example textured surface.

FIG. 5 illustrates a close-up, side view of an example textured surface.

FIG. 6 illustrates a close-up, top-down view of an example textured surface.

FIG. 7 illustrates a close-up, side view of an example textured surface.

FIG. 8A is an optical microscopic image of a silicone texture with mesh over it. FIG. 8B is an optical microscopic image of the silicone texture with mesh removed. FIG. 8C is a scanning electron microscope (SEM) image of the silicone texture.

FIG. 9A is an optical microscopic image of a silicone texture with mesh over it. FIG. 9B is an optical microscopic image of the silicone texture with mesh removed. FIG. 9C is an SEM image of the silicone texture.

FIG. 10A is an optical microscopic image of an exemplary mesh. FIG. 10B is an SEM image of a silicone texture after the mesh is removed.

FIG. 11A is an optical microscopic image of a silicone texture imprinted by multiple mesh layers. FIG. 11B is an SEM image of the same.

FIG. 12A is an SEM image of a silicone texture imprinted by multiple polycaprolactone mesh layers. FIG. 12B is a cross sectional view of the same.

FIGS. 13A-13C are perspective views illustrating steps useful in accordance with certain methods of the present invention which include the provision of a three-dimensional mesh sock to thereby form a textured surface in a breast implant shell.

FIG. 14 shows images of silicone materials made in accordance with different embodiments of the invention.

FIGS. 15A-15D are optical microscopic images and SEM images of a silicone material made in accordance with a method of the invention.

FIGS. 16A-16D are optical microscopic images and SEM images of another silicone material made in accordance with a method of the invention.

FIGS. 17A-17B are SEM images of yet another silicone material made in accordance with a method of the invention.

FIGS. 18A-18B are SEM images of a further silicone material made in accordance with a method of the invention.

### DETAILED DESCRIPTION

As explained above, a number of medical and cosmetic procedures involve the implantation of devices and other objects constructed entirely or partially of silicone, e.g. implants used in breast augmentation and reconstruction procedures, pacemakers, heart valves, artificial joints, etc. In some cases, medical implants can be made of material such as plastic, metal, etc. and at least a portion of the implant coated with silicone as described herein. Although implants made of silicone may be safely implanted in the human body, such implants suffer from a number of problems.

Notably, silicone implants may suffer from a condition known as capsule contracture. When breast implants, or any other objects whether constructed of silicone or another material, are implanted in a patient's body, the body naturally forms a lining surrounding the implant. The formation of this lining, or capsule, is a natural response to the introduction of a foreign object, and the fibrous tissue which forms is similar to scar tissue.

In the case of breast implants, the formation of this capsule may lead to significant complications. In particular, as part of the natural healing process, the body may shrink the fibrous tissue that makes up the capsule, causing the capsule to tighten around the implant. In some cases, this tightening may be significant, altering the implant's shape, appearance, and feel. For instance, the implant may appear to become firmer or harder and may take on a compressed or deformed shape. In addition, capsule contracture may cause problems beyond aesthetic considerations. In some cases, capsule contracture may cause significant pain and discomfort to the patient.

Complications resulting from capsule contracture are the leading cause of patient dissatisfaction with breast augmentation procedures. Accordingly, example embodiments may provide implants designed to prevent complications due to capsule contracture, and procedures for the manufacture of such implants. In particular, some example embodiments may relate to breast implants which may include a silicone shell textured to deter such complications.

It is possible to prevent or lessen the prevalence and severity of complications related to capsule contracture by employing implants having an external surface texture, as opposed to a traditional smooth surface. This is because, the fibroblasts, cells which grow to form the fibrous tissue of the capsule, easily adopt a planar configuration on a flat, smooth surface. Such planar configurations are particularly subject to capsule contracture, and, accordingly, implants which employ a smooth outer shell experience increased rates of problematic contracture.

Certain kinds of textured surfaces, however, may prevent the fibroblasts from forming a planar configuration. Accordingly, in order to prevent the problems associated with contracture, some breast implants, therefore, provide textured external surfaces. For instance, some breast implants have been manufactured which are coated in a polyurethane foam. Such textured surfaces, however, are not ideal as the surface pits which make up the texture are largely isolated from one another, in turn isolating the fibroblasts, and hindering their penetration into the recessed areas of the surface.

Example embodiments described herein, however, provide implants, and procedures for making implants, with interconnected surface pores. In some example embodiments, surface textures are provided with features, e.g. pores and protrusions, which measure in the hundreds of micrometers. Such features are much larger than the cells which form the capsule surrounding the implant. Accordingly, the cells are able to infiltrate the pores of the textured surface, interrupting the formation of a planar capsule configuration. The resulting capsule which does form may be thinner and may be less subject to complicating contracture. To aid this effect, example embodiments may also provide textures with interconnections between the pores formed on the surface. Such interconnections may facilitate the infiltration of cells into the pores furthering the disruption of the planar configuration. Specifically, such interconnections may allow cells to penetrate deep within the surface of the implant by creating an environment in which those penetrating cells are able to easily exchange nutrients and waste. Thus the cells of the capsule may penetrate deeper into the features of the surface than possible in traditional implants. In addition, the interconnected nature of the texture may encourage tissue adhesion to the implant, which may prevent the implant from shifting.

For instance, some example embodiments may provide procedures for making implants comprising at least a portion coated or formed of silicone with textures such as those described above. It is noted that, in the description that follows, example embodiments are described with reference to silicone breast implants. It is to be understood that the scope is not so limited, and other example embodiments apply to other types of implants or devices and to other materials. For example, some example procedures may be used to form other implantable silicone devices, and components of such devices, e.g. pacemakers, artificial joint implants, implants for use in surgical reconstructive surgery, heart valves, coverings for implanted devices, insulation for implanted electrical elements such as pacemaker leads, graft points for implantable devices, etc. In addition, example procedures may be used to create other silicone devices for medical or non-medical purposes, e.g. balloon catheters, tubing, ear plugs and hearing aids, etc. Further, some example embodiments may be used in the creation of devices made of other materials such as, but not limited to plastic or metal coated at least partially coated with silicone.

For the purposes of this application, the terms “strand” and “filament” refer to a single, unitary, elongated structure. The term “fiber” may refer to either a single strand or filament, or to multiple strands or filaments that are braided, coiled, twisted, or otherwise formed into an elongated structure.

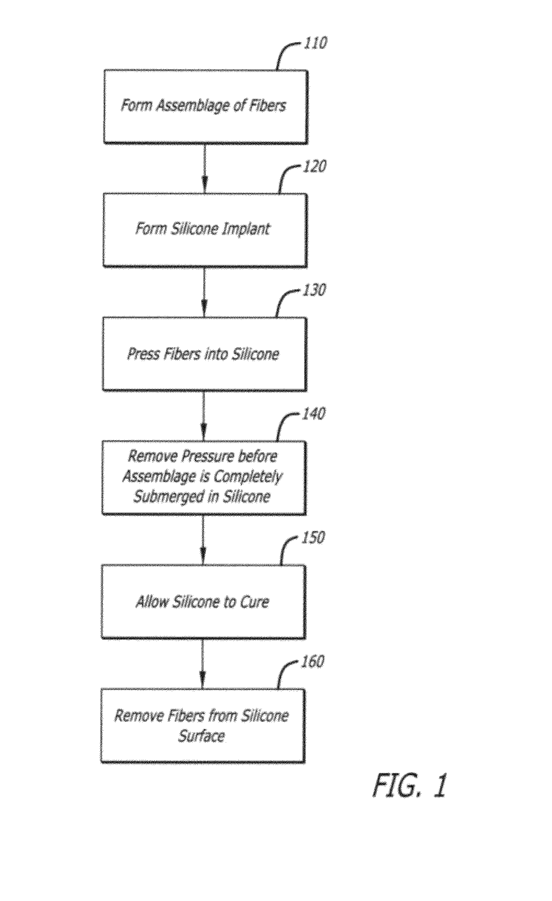

As illustrated in FIG. 1, provided are procedures for the manufacture of silicone breast implants. For instance, example embodiments may provide processes for creating a silicone shell of a breast implant with a textured outer surface, by imprinting a texture using an assemblage of polymer fibers, e.g. a woven cloth, a tangle, a felt, an attached or unattached mesh, or any other structure formed of fibers. Specifically, example embodiments may provide a process in which an assemblage of polymer fibers is impressed into an uncured, or partially cured silicone surface. In such an example process, the silicone may be allowed to cure either fully or partially, after which the fibers may be removed, leaving an imprint of the fibers as a texture on the silicone surface (the silicone need only cure enough to maintain the imprinted structure when the fibers are removed). Such example processes may be capable of providing silicone implants with desirable surface textures without sacrificing the mechanical properties of the silicone, e.g., the hardness, tensile strength, elongation, tear strength, and abrasion resistance of the material.

As illustrated at block 110, an assemblage of polymer fibers may be formed which will be used to imprint a texture on a silicone surface of an implant. The fibers may be constructed of any material with suitable mechanical strength and flexibility. In some example embodiments, the polymer employed may need to be relatively insoluble in either xylene or toluene, but may be soluble in other organic solvents such as methylene chloride and chloroform. For example, biodegradable materials such as Vicryl 910, poly (L-lactic acid-co-trimethylcarbonate), polycaprolactone, poly(methyl methacrylate), poly(L-lactic acid), poly(lactic-co-glycolic acid), and the like, may be used to construct the assemblage of polymer fibers. It is noted that the materials listed above as example materials have all been approved by the FDA for use in therapeutic applications.

Other degradable polymers that can be used to form the fibers include, but are not limited to poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid or combinations thereof.

Such materials may be capable of forming fibers of sufficient strength and may be easily removed from the cured (or partially cured) silicone, e.g., by dissolution in an appropriate solvent or through hydrolytic degradation. Any other materials which may be removed from silicone through a process which does not damage the silicone surface may be used as well. For instance, other materials which are soluble, or otherwise removable, by a substance which does not significantly affect silicone may be used. Materials with melting or sublimation points below that of silicone, or which are otherwise weakened by the application of heat, may also be used. In such cases, the removal process may include heating the silicone surface to a temperature at which the mesh may be removed from the surface. Materials which degrade through other processes may also be used. For instance, photosensitive materials may be used which may undergo a change in the presence of light, or a particular wavelength of light, which may allow for removal.

The assemblage itself may be formed from the polymer fibers in any reasonable manner. For instance, one or more polymer fibers may be woven or knit together to form a mesh. In other example embodiments fibers may be bundled together and pressed to form a felt or mat structure. In other embodiments, fibers may be twisted together before being formed into a mesh, felt, or other structure. Any other combination of fibers may also be used, whether having an intentional structure, or a structure randomly formed. In addition, the fibers need not be formed into an assemblage before use. Rather, in some example embodiments, individual fibers may be deposited onto a silicone surface which is to be imprinted (either randomly or according to some pattern).

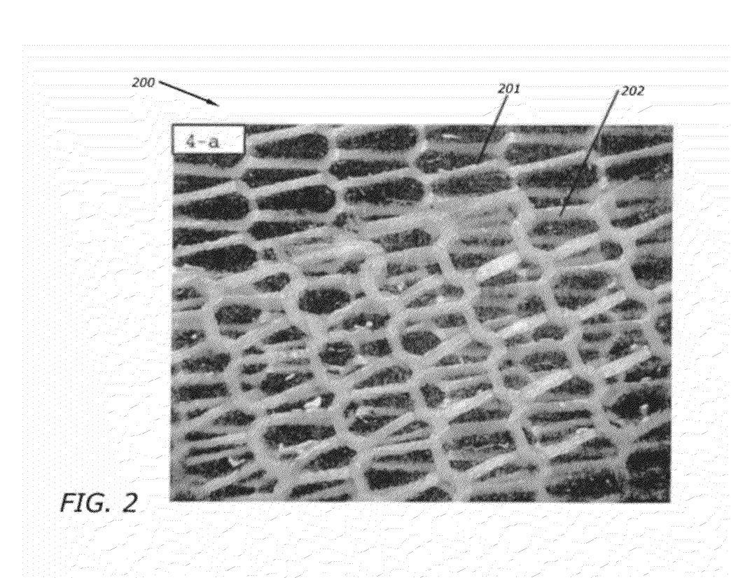

For instance, an example mesh is illustrated in FIG. 2. As shown in the figure, the mesh 200 may be woven into a pattern. The woven mesh 200 may have the form of any traditional woven material. For instance, a fiber 201 may be linked together with itself or other fibers to create a mesh 200. Thus, in some places, fibers 201 in the weave may overlap one another. In addition, open spaces (or eyes) 202 may exist between the fibers 201 of the mesh 200. These structures may naturally form a texture of interconnected pores when imprinted in a silicone surface. For instance, where the fibers 201 overlap the thickness of the mesh 200 may be the greatest, ultimately resulting in a pore formed in the imprinted surface. These pores may be interconnected, as the fibers 201 that make up the woven mesh may continue beyond each area of overlap. In addition, where the mesh is open, it will not displace the silicone material, and, therefore, a protrusion may be formed on the surface. Of course other fiber structures, e.g. a felt or mat structure, may also be used, as described above.

In some example embodiments, a single polymer filament may form each fiber 201 of the mesh 200 (or other kind of assemblage). In such example embodiments, the silicone texture which may arise from the mesh 200 may have a smooth channel structure. This channel structure may encourage the growth of fibroblasts within the channels and pores by facilitating the transport of nutrients and waste products into and out of the features of the texture. In other example embodiments, multiple filaments may be used to form each fiber 201 of the mesh 200. Such embodiments may result in channels having a thread pattern imprinted in the silicone surface of the channel. These thread patterns may facilitate cell attachment. In some example embodiments single filament and multiple filament fibers may be used in combination to provide the desired properties. In such example embodiments, some of the resulting channels formed in the silicone may contain thread patterns while other channels may be smooth. In addition, some example meshes, or other fiber structures, may be formed using two or more fibers 201 of different widths, compositions, or other properties, which may be woven or otherwise formed together.

Once the mesh 200, or other assemblage of fibers, is formed, it may be strengthened before use. For example, once the fibers 201 are woven together into a formed mesh 200, or other structure, the fibers 201 may be joined together, e.g. through the application of heat, pressure, etc., fixing the fibers together. In other embodiments, an adhesive may be applied which may cause the fibers 201 to adhere to each other. In this way the mesh 200 may be strengthened and the pattern of the mesh 200 may be made resilient. In addition, as will be described more fully below, contact between fibers 201 of the mesh may be important to the removal process, and may be encouraged through application of heat, pressure, adhesive, etc.

The physical properties of the mesh 200 itself, or other assemblage of fibers, affect the texture that is ultimately formed in the surface, e.g. the pattern of the mesh 200, the thickness of the polymer fibers 201, the size of the eyes in the mesh 202, etc. These characteristics may be chosen as appropriate for the intended application. For instance, the average thickness of the fibers 201 may be between 100 and 1000 μm. This thickness refers to the diameter of each fiber 201 of the mesh 200, or other assemblage. In addition, the average size of the eyes 202, appearing in the mesh may also be chosen. For instance, on average the eyes 202 appearing in the mesh 200 may have a size in the range of approximately 100×100 μm to 2000×2000 μm. Of course, the eyes 202 may have any reasonable shape, and the sizes suggested represent areas rather than dimensions of the boundaries of the eyes 202. It is also noted that not all of the eyes 202 need to have the same size. Rather, the pattern of the mesh 200 may form eyes 202 of differing sizes. Other characteristics of the mesh of other assemblage may also be chosen.

In some example embodiments, multiple layers of mesh 200, or of another fiber structure, may be joined together to form a single mesh 200 or structure used to imprint a silicon surface. For instance, a mesh 200 may be formed which is itself formed of two or more polymer meshes 200 stacked together. In such embodiments, the layers may employ the same mesh pattern and other characteristics, or different mesh 200 layers may have different properties. Further different combinations of fiber structures may be used. For instance, a woven mesh structure may be layered with a felt layer, or two felt layers may be used, etc. Again, the final assemblage may be formed through a strengthening process. For instance, layers of mesh 200 may be stacked together, and together be subjected to a strengthening process (e.g. the application of heat, pressure, adhesive, etc.), interconnecting the meshes 200, to form a single final structure.

Returning to FIG. 1, as illustrated at block 120, in example embodiments a silicone shell, or a portion of the shell (e.g. one half of the final shell) or a silicone coating, may be formed from uncured or partially cured silicone. Of course any other object which will be imprinted may also be formed at this point. Any traditional process, e.g. a molding process, may be used to form the silicone into an appropriate shape for the implant. In addition, it is noted that the entire silicone object need not be constructed at once. For instance, the shell, or other silicone surface, may be constructed in layers, e.g. a silicone layer may be formed and allowed to at least partially cure, after which another layer of silicone material may be applied, etc. In such a case, less than the total number of layers may be imprinted.

Once the silicone shell is formed, and before it has cured (though the silicone may be partially cured at this point), at block 130, an assemblage of fibers may be pressed into the surface of the silicone (or individual fibers may be deposited on and pressed into the surface). For example, the assemblage may be placed onto the silicone surface, in a location on the surface that is to be imprinted with a texture. Because the silicone is uncured or partially cured, the fibers may penetrate into the silicone layer. In some example embodiments, external pressure may be applied tending to push the fibers into the silicone layer. For example a mechanical press may be used to force the fibers into the uncured or partially cured silicone layer.

In order to imprint the silicone with a texture, the fiber assemblage may not be completely submerged in the silicone, because if the assemblage were to become completely submerged the silicone might form a smooth surface over the submerged assemblage (embedding the assemblage in the silicone object). Rather, at least a portion of the fiber structure may remain unsubmerged in the silicone. Thus, at block 140, in some example embodiments, pressure is not applied to the assemblage once it has been pushed partially into the silicone surface. By discontinuing the applied pressure before the assemblage is settled entirely into the silicone layer, the process may ensure that the final surface is textured, e.g. that the surface has openings reflecting the structure of the assemblage of fibers. In other embodiments, however, the assemblage may be pressed entirely into the silicone material. In such embodiments, the process of creating a texture may include removing a portion of the silicone material after it has cured in order to expose the assemblage. Further, in some example embodiments, portions of the assemblage of fibers may be completely submerged in the silicone. In such cases, tubes may form in the resulting texture.

In some example embodiments, a single, unitary assemblage of fibers need not be used to imprint the silicone object. For example, multiple patches (of fiber assemblages) may be used to imprint the silicone surface. In such cases, more than one individual assemblage may be pressed into the uncured or partially cured silicone surface. These patches may have the same characteristics as each other, or different patches may have different characteristics. In addition, the patches may be applied in an overlapping or a non-overlapping manner.

In example embodiments, where the patches are applied in an overlapping manner, the patches may be pressed into the silicone surface independently such that at least some of the patches used partially overlap along an edge of the patch (although the patches need not overlap at all). In such examples, the patches may remain entirely distinct throughout the imprinting process. In other examples, however, the overlapping sections of the patches may be joined together. For instance, some example embodiments may include heating, or applying pressure to, the patches, causing the patches to join together where they overlap. Alternatively, example embodiments may include applying an adhesive which may join the patches to each other, or may include stitching the patches together.

Once the fiber assemblage has been pressed into the silicone object, the silicone may be allowed to cure, in block 150. The curing processes may be a traditional curing process. During this curing process the silicone may harden into its final form. Because the assemblage of fibers is imbedded in the silicone during the curing process, the silicone may harden into a form accommodating the shape of the imbedded fibers. The silicone may be allowed to cure until the curing process is complete. Alternatively, the silicone may be allowed to partially cure before continuing the process. In some embodiments, the silicone is cured using constant heating or a particular ramping temperature program.

Once the silicone is completely or partially cured, at block 160, the assemblage of fibers may be removed from the silicone surface. Here the fibers may be removed using any reasonable process which does not damage the silicone structure. Such processes may depend on the choice of polymer used for the fibers. For instance, in some example embodiments, a solvent may be applied to the surface, in which the polymer of the fibers is soluble, while the silicone is insoluble. In such embodiments the fibers may be dissolved entirely away, leaving behind the silicone object. In other example embodiments, other processes may be used to remove the fibers. For instance, the fibers may be removed hydrolytic degradation in some example embodiments. In other embodiments, the fibers may be removed through the application of heat, melting the polymer, or through the use of a physical retracting force.

Once the fibers are removed, the silicone surface which remains may be imprinted with a texture, e.g. the pattern of a mesh used. Thus the texture of the surface may have a structure which is a negative of the structure of the imprinting fibers. Accordingly, the fibers of the assemblage used may leave pores and channels in the silicone surface, while other areas of the assemblage, e.g. the eyes of a mesh, may leave surface protrusions. In this way a silicone surface may be formed with channels, pores, and other structures which may together form a texture which effectively prevents the cells, which will eventually grow over the surface when implanted, from taking a planar configuration.

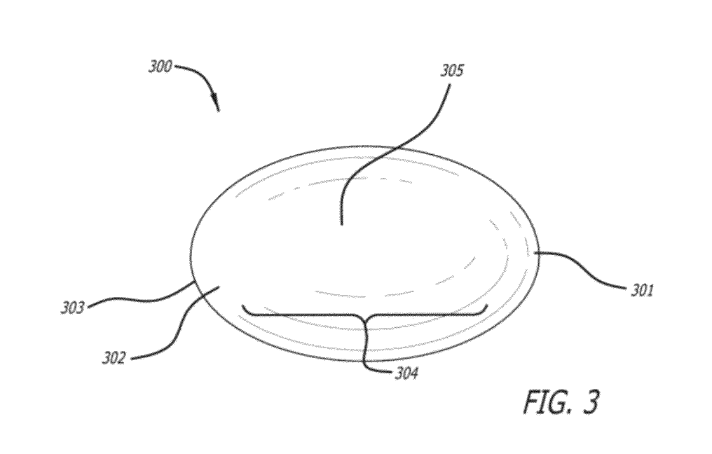

Some example embodiments may also provide implantable objects and devices having an external surface constructed partially or entirely of silicone imprinted with an assemblage of polymer fibers, e.g. a mesh. For instance, FIG. 3 illustrates an example breast implant 300. As shown in the figure, the implant 300 may include an outer shell 301. This outer shell 301 may be formed of any suitable material, for instance, silicone. Any other suitable material may also be used, however. The outer shell 301 may have both an inner 302 and an outer surface 303. The outer surface 303 may be a surface which is exposed to a patient's body when the implant 300 is in use. The inner surface 302 may define an internal cavity 304 which does not come into contact with the patient's body. This outer shell 301 may be constructed of a single piece of silicone material, or multiple pieces. For instance, the shell 301 may be constructed out of two halves, which may be attached to each other to form a single shell 301 during the manufacturing process, using any reasonable connection technique (such as the application of additional silicone material). In embodiments employing multiple pieces, each of the pieces may be individually textured (or partially textured), for example using the imprinting technique described above. In other embodiments, however, the pieces need not all have an imprinted texture.

The implant 300 may also include a filler material 305. For instance, as explained above, the shell 301 may form an internal cavity 304. For example the shell 301 may be shaped as a closed bag. This cavity 304 may be filled with a filler material 305 which may give the implant 300 volume and shape. The filler material 305 may be any suitable material. For instance, the filler material 305 may be a saline solution, or a silicone gel, or some other suitable material.

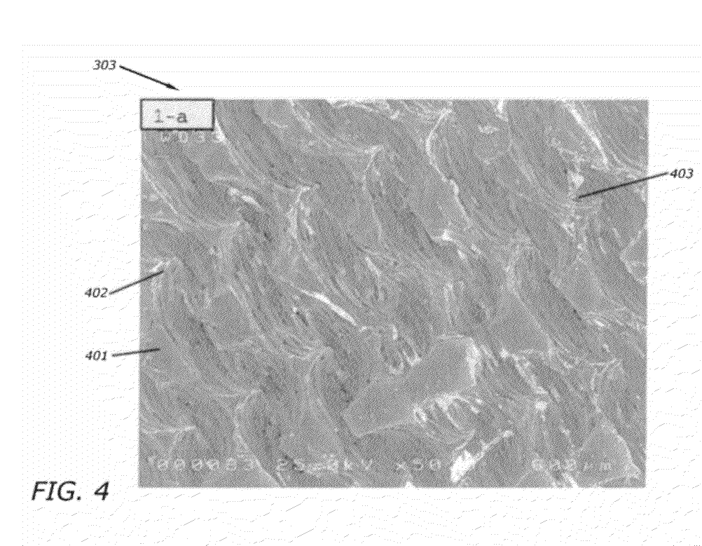

In example embodiments, all or part of the outer surface 303 of the shell 301 may be imprinted with a texture 306 designed to alleviate the complications associated with capsule contracture. For instance, FIG. 4 provides a close-up, top-down, illustration of the outer surface 303 of an example implant 300. As can been seen in the illustration, the surface 303 may be textured, rather than smooth. As illustrated, a repeating pattern may be formed in the silicone surface 303. The pattern may be formed in the silicone material of the surface itself, for example using an imprinting technique like that described above (e.g. employing a mesh of fibers). In other example embodiments, the pattern formed in the silicone material of the surface, need not have a repeating pattern. For instance, the pattern may have a random structure, having been imprinted with an assemblage of fibers having a felt like structure.

In particular, the surface may include a number of structures, including pores 401, protrusions 402, and channels 403. For instance, such structures may be formed by the pattern of an assemblage of fibers used to imprint the surface 303. Thus, the pores 401 and channels 403 may represent the portions of the surface which cured around a polymer fiber, while the protrusions 402 may represent areas of the surface where no fiber was present during the curing process, e.g. inside an eye of a mesh. Such a pattern may provide a series of interconnected pores 401, which may serve to prevent the formation of a planar configuration of capsule cells when the implant 300 is in use. Unlike in traditional implants, the interconnectedness of the channels 403 and pores 401 may enhance the disruption of such a planar configuration by encouraging cell growth in the recessed areas of the textured surface 303. The texture pattern may have any configuration, e.g. size, pattern shape, etc., and may be a repeating pattern or may have a changing structure. In addition, it is noted that pores and channels need not be distinct features. Rather the pores and channels may simply refer to recessed features formed on a surface.

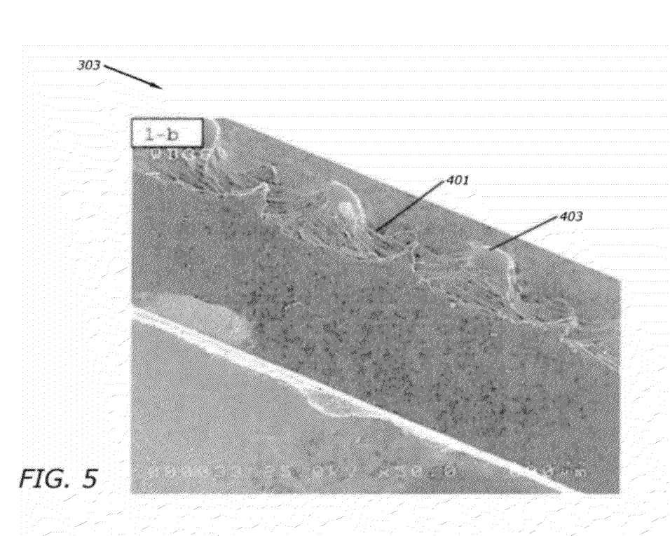

The differing elevations of the surface 303 may be seen more clearly in FIG. 5 which provides a view of an example surface 303 from the side. As seen in FIG. 5, the surface pattern has both high and low points. As explained above, the raised areas may reflect the open areas of an assemblage of fibers (e.g. eyes of a mesh) which was used to imprint the surface, while the lower features may reflect places in which the fibers were pressed into the surface material during the curing process. Here the relative difference in elevations of the various features may be determined by the thickness of the assemblage used, the thickness of the polymer fibers, the number of fibers, whether multiple layers were used, how deeply into the surface the assemblage was pressed, etc. In some example embodiments, the average distance between the highest and lowest points in the pattern may be in the range of about 100 μm to about 500 μm, or about 100 μm to about 1000 μm. Such elevation changes may be sufficient to disrupt the formation of a planar capsule cell configuration.

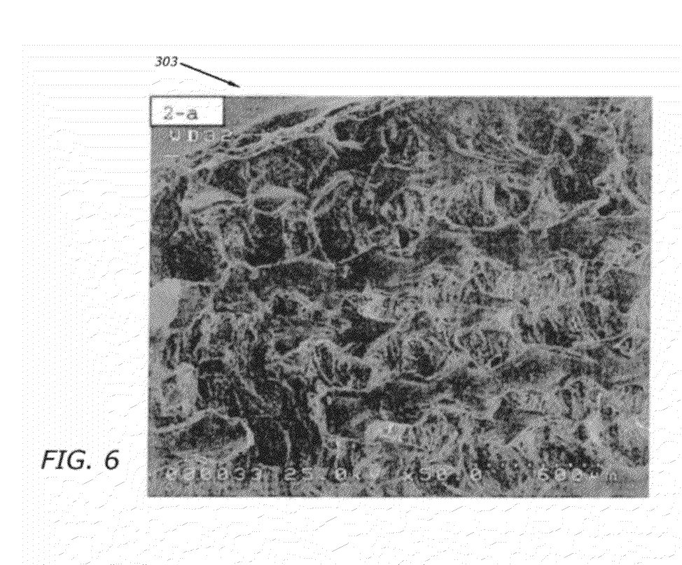

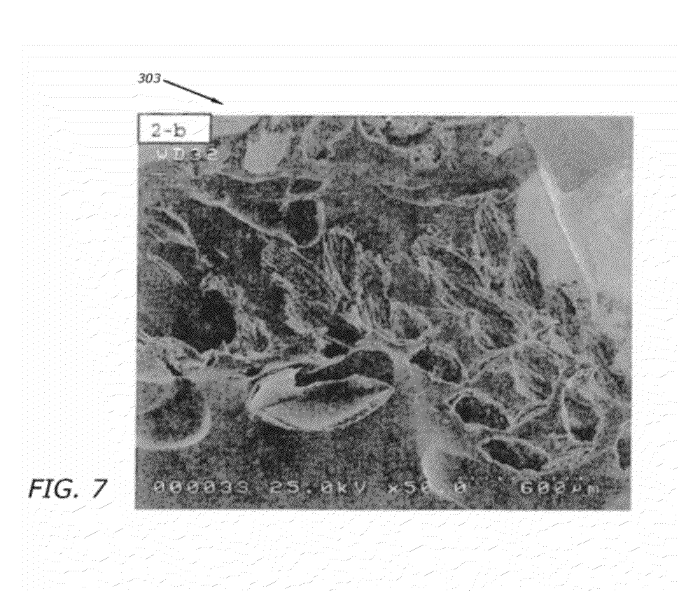

As noted above, multiple layers of assembled fibers may be used to imprint the surface 303. In such embodiments, the pattern formed on the surface 303 may be more complicated than the simple pattern depicted in FIGS. 4-5. For example, FIGS. 6-7 illustrate an example surface imprinted with three layers of mesh. FIG. 6 provides a top-down view of the pattern formed by the layers of mesh, and FIG. 7 shows the corresponding side view. As can be seen in the figures, the pattern imprinted in the silicone may again include a series of pores 401, protrusions 402, and channels 403. Here again the structures may be of a size sufficient to discourage formation of a planar cell configuration in the capsule. However, the pattern may be more complicated than in the case of a single mesh (or other assemblage of fibers). For instance, structures may be formed on the surface 303 at an intermediate height.

It is also noted that, in some embodiments, portions of the imprinting fibers may become completely submerged in the silicone surface during the imprinting process. When the fibers are removed, e.g. through dissolution of the polymer fibers, tubes may be formed in the surface. Such tubes may have openings to the surface where the polymer fibers entered and exited the silicone during the curing process. Thus some example implants 300 (or other devices) may have textured surfaces 303 including tubes formed through the surface 303.

### Example 1

### Preparation of Silicone Texture Imprinted by One Layer of Osteoprene Mesh

A osteoprene mesh (poly(L-lactic acid)-co-trimethylenecarbonate mesh) is provided. The mesh was custom-made by Poly Med Inc. (Anderson, S.C.). It had an open hole, or pore size, of 485×195 μm and a filament thickness of 285 μm.

To prepare a silicone texture imprinted by osteoprene mesh, first, to a 3″×3″ mold, 20 mL of MED 6400 dispersion was added. MED 6400 is a high temperature vulcanization (HTV) silicone available in 36 wt % of xylene (Nusil Technology, Santa Barbara, Calif.). Then, the mold with silicone dispersion was placed into a fume hood for 8 hrs to allow the xylene to evaporate. A 2″×2″ osteoprene mesh was then placed on the surface of the above mentioned uncured silicone. A flat spatula was used to push the mesh to uncured silicone. The above mentioned silicone, embedded with the osteoprene mesh, was then cured at a heating profile of 75° C. for 45 minutes, 150° C. for 2 hours, and 165° C. for 30 minutes. After cooling down to room temperature, the silicone-osteoprene composite film was peeled off. The composite film was placed into methylene chloride allowing the osteoprene mesh to dissolve. The swollen silicone film was placed in a fume hood for a couple of hours, then heated at 126° C. for 1 hour. The textured silicone was examined by optical microscope and SEM.

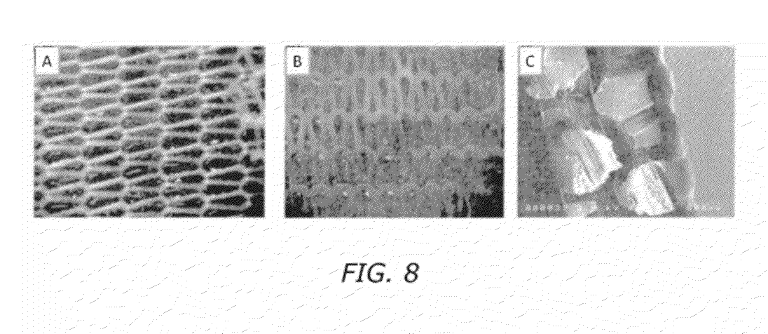

FIG. 8A is an optical microscopic image of the silicone texture with the osteoprene mesh. FIG. 8B is optical microscopic image of the silicone surface texture after the osteoprene mesh was removed. FIG. 8C is an SEM image of the resulting silicone surface texture.

### Example 2

### Preparation of Silicone Texture Imprinted by Two Layers of Osteoprene Mesh

Mesh materials and silicone were described in Example 1. The process for making textures was similar to the process described in Example 1, except that two layers of osteopyrene mesh were used to create more sophisticated texture. The textured silicone was examined by optical microscope and SEM.

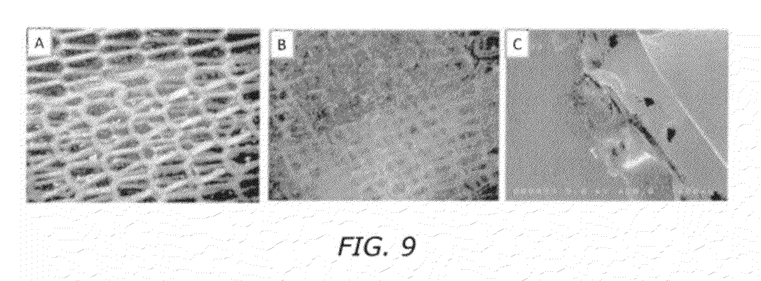

FIGS. 9A, 9B and 9C are optical microscopic and SEM images of silicone texture imprinted by two layers of osteoprene mesh (open hole, 485×195 μm; thickness, 285 μm). FIG. 9A is an optical microscopic image of the silicone texture with osteoprene mesh present. FIG. 9B is an optical microscopic image of the silicone surface texture after osteoprene mesh was removed. FIG. 9C is an SEM image of a cross section of the resulting silicone texture.

### Example 3

### Preparation of Silicone Texture Imprinted by Osteoprene Mesh with Different Pore Size and Filament Thickness

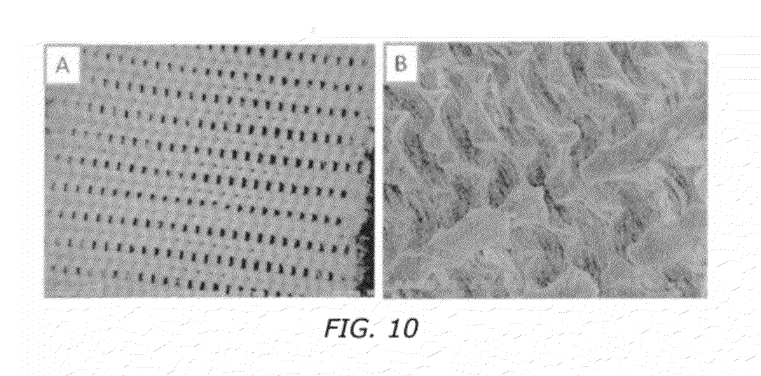

An osteoprene mesh with a pore size, 252×156 μm and filament thickness of 400 μm is provided to make a silicone texture using the procedure described in Example 1. FIG. 10A is an optical microscopic image of the silicone texture imprinted by the osteoprene mesh. FIG. 10B is an SEM image of the silicone surface texture after the osteoprene mesh was removed.

### Example 4

### Preparation of Silicone Texture Imprinted by Multi Layers of Osteoprene Mesh by Controlled Wetting Process

To prepare a silicone texture imprinted by osteoprene mesh, first, to a 3″×3″ mold, 20 mL of MED 6400 dispersion was added. Then, the mold with silicone dispersion was placed into a fume hood for 8 hrs to allow the xylene to evaporate. Then, four layers of 2″×2″ osteroprene mesh (open pore size: 485×195 μm; Filament thickness: 285 μm) were placed onto the surface of uncured silicone. Pressure was applied to allow four layers of osteroprene mesh to attach tightly. Then, 4 mL of xylene were added to the silicone to allow the silicone to wet mesh. After the xylene was evaporated, the silicone was cured according to heating profile of Example 1. After the temperature was cooled to room temperature, the mesh material was removed using methylene chloride and the resulting textured silicone was dried.

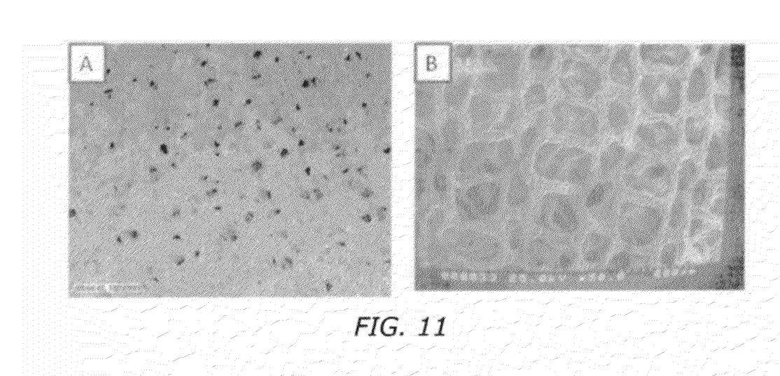

FIG. 11A is an optical microscopic image of the silicone texture imprinted by multi layers of the osteoprene mesh and FIG. 11B is an SEM image of the same.

### Example 5

### Preparation of Silicone Texture Imprinted by Polycaprolactone (PCL) Mesh

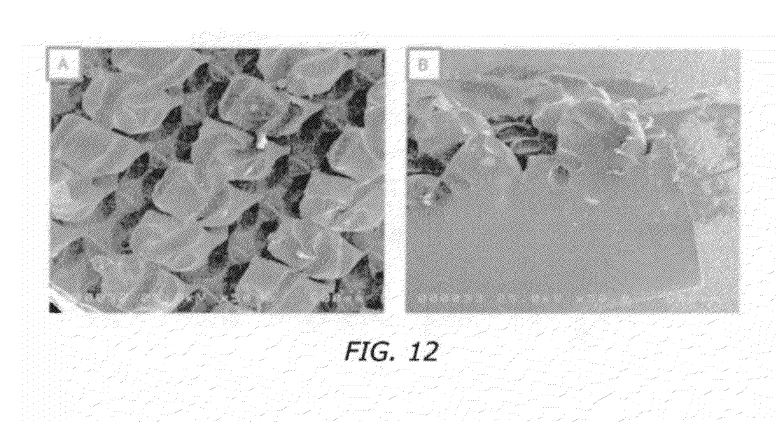

A silicone texture using polycaprolactone mesh with pore size, 539×625 μm and filament thickness of 340 μm was prepared according to the method of Example 1. FIG. 12A is an SEM image of the top of the silicone texture imprinted by two layers of the polycaprolactone mesh. FIG. 12B is a cross-sectional SEM image of the same.*****

In another aspect of the invention, a method of making a porous material in a contoured shape is provided. The method generally comprises the steps of a) applying a matrix material to a contoured mold, applying a fibrous covering to the matrix material on the mold, treating the matrix material having the fibrous covering to cure or harden the matrix material on the mold, and removing the fibrous covering from the cured or hardened matrix material, wherein fibrous covering removal results in a porous material.

The fibrous covering may comprise a fiber assemblage that is generally in the shape of a three-dimensional covering, for example, shape having an open end for fitting onto a mold, and a closed end. The fibrous covering may be in the form of, for example, a tube, or a sock, or other shape corresponding to the shape of a contoured mold. For example, as shown in FIGS. 13A and 13B, the fibrous covering 1200 may be in the form of a mesh “sock” with a string 1202 or other suitable means for cinching or tightening the sock onto a contoured mold surface 1224 of a mold assembly 1230. Alternatively, a relatively less fitted sock 1240 may be simply wrapped around the mold surface 1224 as shown in FIG. 13C. The fiber assemblage can be a woven structure made of any of the materials described elsewhere herein which can be removed from the matrix material.

The essence of this embodiment is that porous materials, for example, silicone materials with porous structures, are created by using one or more layers, for example, multilayers, of removable polymeric socks. The process for making porous silicone, with a base layer, involves: 1) wrap multilayers of polymer socks onto a mandrel with a silicone base layer, 2) dip the mandrel, with wrapped socks, with silicone dispersion, 3) cure silicone, and 4) remove socks by solvent dissolution. This process allows creation of an integral 3D silicone shell with the characteristics such as being relatively easy to fabricate in a desired shape without involving any lamination or adhesion process.

Breast implant shells may be made on a mandrel, using multilayers of polymer socks, e.g. socks comprising woven fibers of poly(L-Lactic acid), PLLA. The bare mandrel, prior to any application of silicone dispersion, is wrapped with a layer of PLLA sock, which is tightened by a thread at the neck of the sock. A second layer of PLLA sock is put onto the first layer of sock and tightened. The wrapping of socks is repeated until multilayer of socks are assembled. (It is to be appreciated that although in this example, the socks are applied to a bare mandrel, in other embodiments, the mandrel may first be contacted with or coated with a silicone dispersion prior to application of the socks.)

The mandrel which is assembled with multilayer of PLLA socks is then dipped into silicone dispersion, e.g. 25 wt % of PN-3206-1 in xylene, and placed on a mandrel stand to allow xylene to evaporate. The mandrel with silicone-coated multilayer socks is placed into an oven at a certain temperature for predetermined time to cure the silicone. The socks are then removed by soaking the cured silicone-socks composite into an organic solvent, e.g. methylene chloride, chloroform, acetone, or tetrahydrofuran.

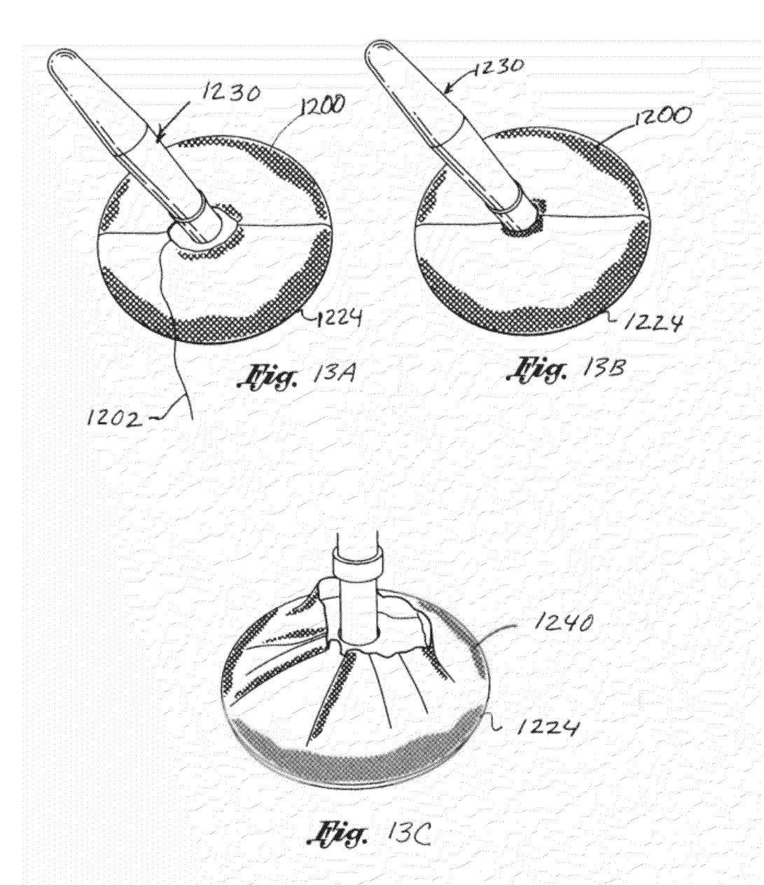

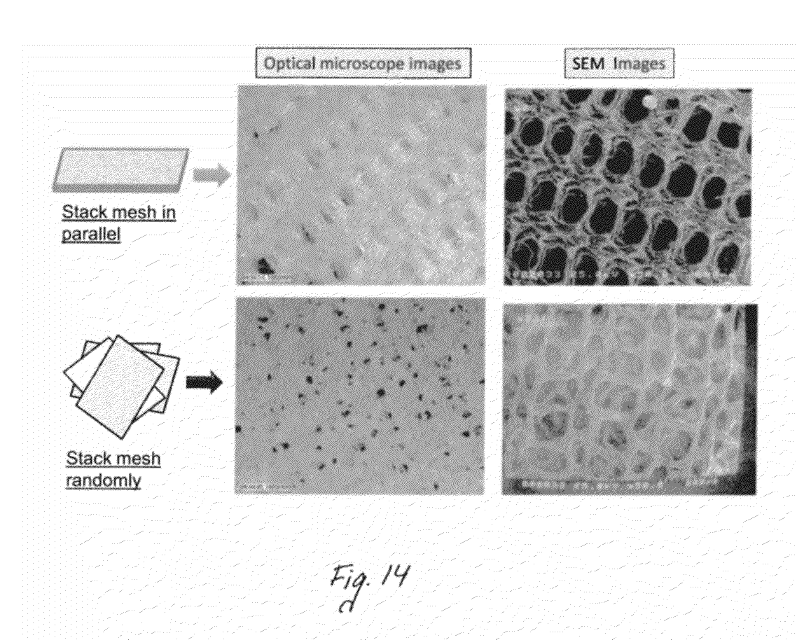

The porosity, pore size, and pore interconnections are controlled by the parameters of meshes or socks, e.g. mesh open pore size and filament thickness, and the means of stacking meshes. Parallel stacking socks will create a channel-like porous structure, random stacking, however, will create a more randomly interconnected porous material. This concept is illustrated in FIG. 14 which shows Optical Microscope and SEM images of porous silicone based materials made using a method of the invention as described herein.

As shown, parallel stacking of multilayers of meshes creates a relatively channel-like silicone material. Random stacking meshes of multilayers of meshes creates a more interconnected material.

### Example 6

### Preparation of a 3D Silicone Porous Material Using 3 Layers of Poly(L-Lactic Acid) (PLLA) Socks

A three dimensional elastomeric textured device, useful as a component of a breast implant, was made as follows.

Procedures:

A conventional mandrel for forming a breast implant shell was dipped with 35% silicone and cured at 126° C. for 1 hour and 25 minutes. The mandrel with cured silicone was dipped again with 35 wt % silicone, then placed into a fume hood to allow xylene to evaporate.

Three layers of PLLA socks are applied, one-by-one, onto the mandrel with silicone base layer. The PLLA socks had open pore size of about 500 μm×500 μm and filament thickness of about 500 μm.

The mandrel with the multilayer of PLLA socks was dipped with 25% 3206 silicone, followed by cured at 126° C. for one hour and 25 minutes. The mandrel was allowed to cool to room temperature. Then it was dipped with 15% 3206 silicone, and cured at 126° C. for 1 hour and 25 minutes. Silicone PN-3206-1 is available in available in 35 wt % of xylene dispersions from Nusil Technology, Carpinteria, USA. The dispersion of silicone is diluted with xylene to get a concentration of 25 wt %, 20 wt %, and 15 wt % respectively.

The mandrel with cured silicone-PLLA sock composite was soaked in chloroform for one time, then in methylene chloride twice, and finally dried in the fume hood.

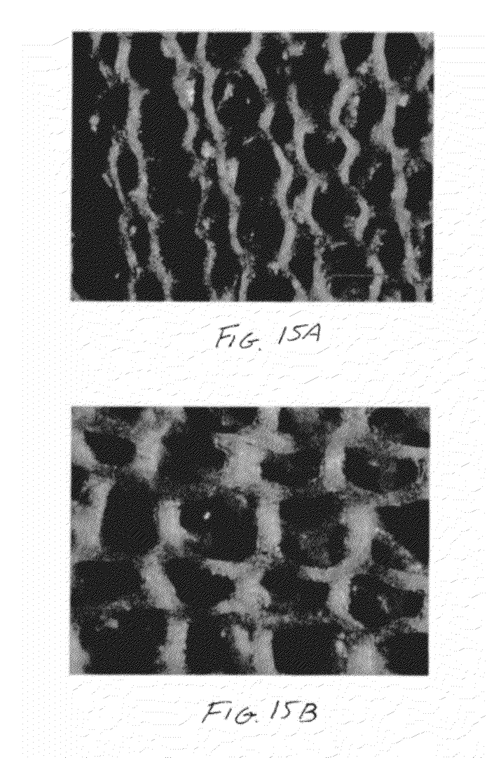

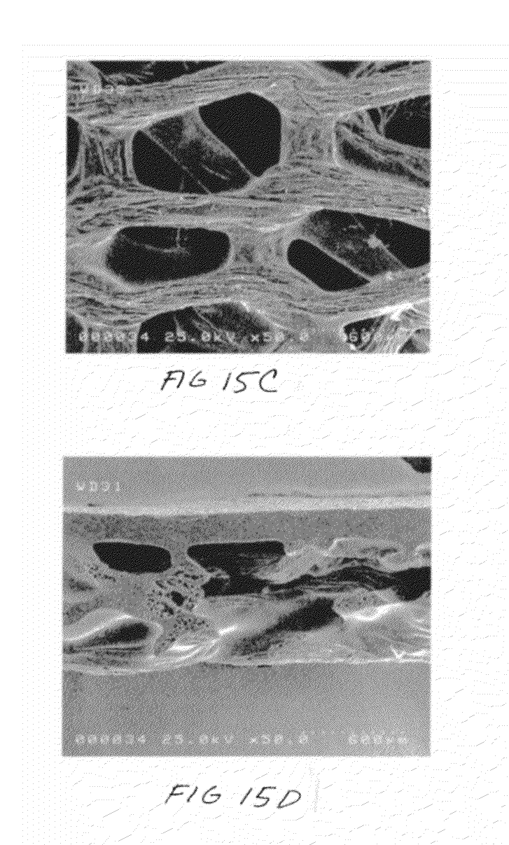

Optical microscopic images of the resulting silicone porous material are shown in FIGS. 15A and 15B. Optical microscopic images of silicone: (A) 20×, (B) 30×(open pore, 500×500 μm; thickness, 500 μm). SEM images of the porous material are shown in FIGS. 15C and 15D, top view and cross section respectively.

### Example 7

### Preparation of a 3D Silicone Porous Material Using 6 Layers of Poly(L-Lactic Acid) (PLLA) Socks

The same materials and procedure are followed as in Example 6, except instead of three layers of PLLA socks, 6 layers of PLLA socks are applied to the mandrel.

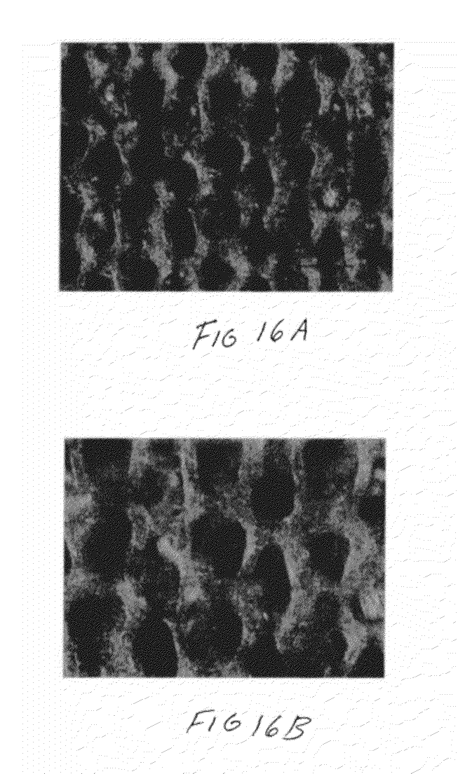

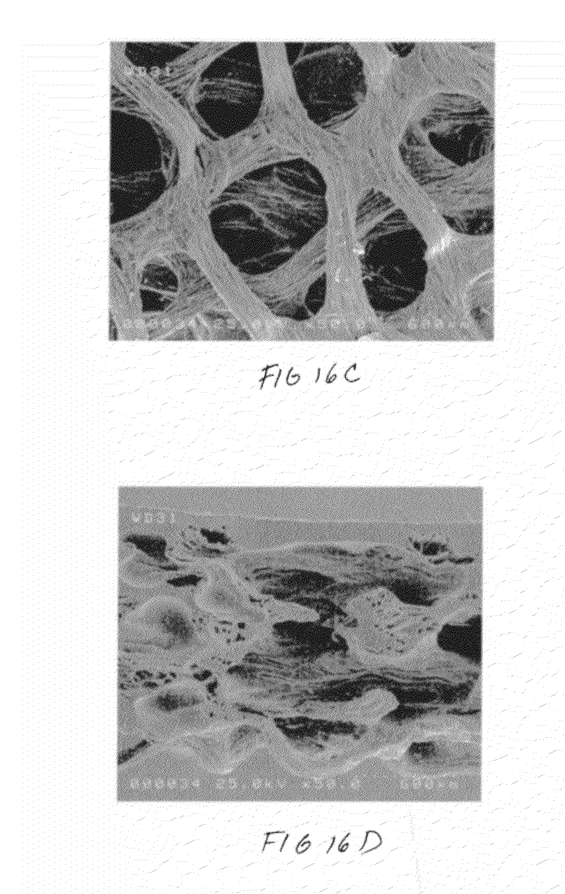

Optical microscopic images of the resulting silicone porous material are shown in FIGS. 16A and 16B. SEM images of the porous material are shown in FIGS. 16C and 16D, top view and cross section respectively.

### Example 8

### Preparation of Flat Silicone Foam-Like Material with a Silicone Base Layer by Osteoprene Mesh (Poly(L-Lactic Acid)-Co-Trimethylenecarbonate Mesh)

Osteoprene mesh (poly(L-lactic acid)-co-trimethylene carbonate mesh having pore size 484×508 μm; filament thickness, 335 μm was made by Poly_Med Inc. 6309 Highway 187, Anderson, S.C. 29625.

Silicone PN-3206-1 is available in 35 wt % of xylene dispersions from Nusil Technology, Carpinteria, USA. The dispersion of silicone is diluted with xylene to get a concentration of 25 wt %, 20 wt %, and 15 wt % respectively.

A few round pieces of osteoprene mesh (485×195) with a diameter of 110 mm were cut, stacked, layer by layer, and placed into a positive pressure filter. About ml of 35% PN-3206-1 was poured onto the top layer of the stacked meshes. Positive air pressure to remove excessive amount of silicone was applied. The silicone-coated meshes were placed onto an uncured silicone base layer and placed into an oven.

The silicone was cured at 80° C. for 4 hrs and 126° C. for 1 hour and 25 minutes to get silicone-mesh composite. The mesh was removed by soaking the composite into methylene chloride, with solvent change for three times. The silicone foams were dried in a fume hood.

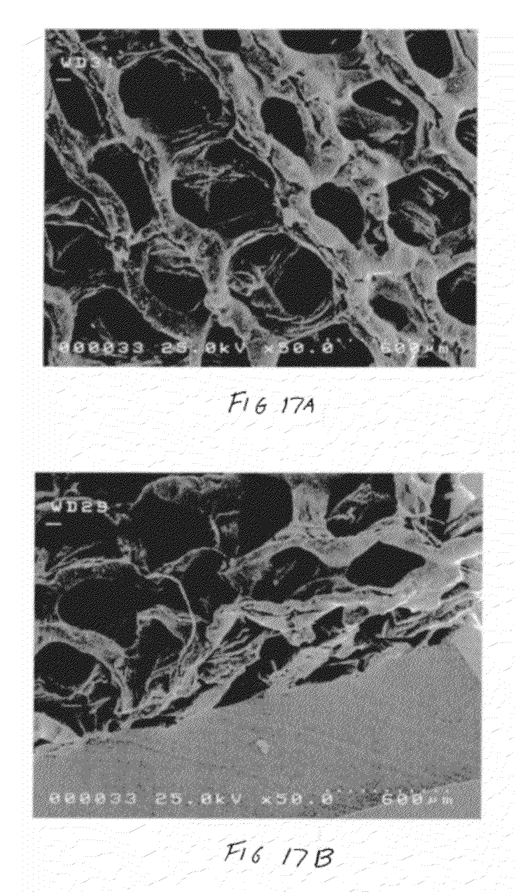

FIGS. 17 A and 17 B are SEM images, top view and cross sectional view, respectively, of the resulting porous silicone material made as described in this example, the materials having a thickness of about 600 μm.

### Example 9

### Preparation of Flat Silicone Foam-Like Material Using Osteoprene Mesh (Poly(L-Lactic Acid)-Co-Trimethylenecarbonate Mesh)

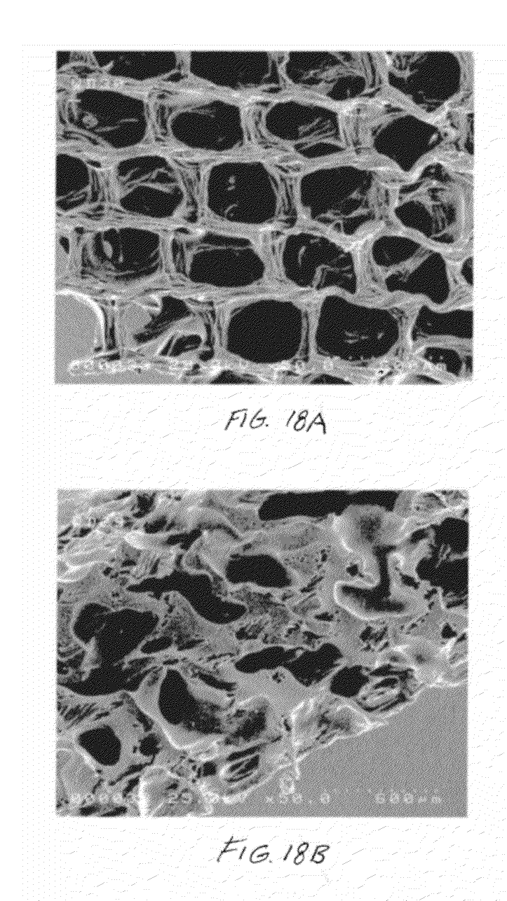

The procedure for preparing a flat silicone foam is identical to Example 8 except that the materials were prepared in a free-standing form and no base layer was used. FIGS. 18A and 18B are SEM images, top view and cross sectional view, respectively, of the resulting porous silicone material, the material having a thickness of about 1500 μm.

In the preceding specification, the present invention has been described with reference to specific example embodiments thereof. It will, however, be evident that various modifications and changes may be made thereunto without departing from the broader spirit and scope of the present invention. The description and drawings are accordingly to be regarded in an illustrative rather than restrictive sense.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method of making a textured component of a breast implant shell, the method comprising the steps of:
providing a mandrel;
applying a polymeric mesh sock to the mandrel;
contacting the sock with a silicone dispersion;
at least partially curing the silicone dispersion to form a silicone elastomer with the sock in contact therewith; and
after the silicone is at least partially cured, removing the sock from the silicone elastomer to thereby form a textured component of a breast implant shell.

2. The method of claim 1 further comprising applying at least one additional polymeric mesh sock onto the mandrel prior to the step of at least partially curing.

3. The method of claim 1 wherein the step of contacting the sock with a silicone dispersion is performed after the step of applying the sock.

4. The method of claim 1, wherein the sock is comprised of at least one of Vicryl 910, poly (L-lactic acid-co-trimethylcarbonate), polycaprolactone, poly(L-lactic acid), poly(methyl methacrylate) and poly(lactic-co-glycolic acid).

5. The method of claim 1, wherein the sock is comprised of poly(L-lactic acid.

6. The method of claim 1, wherein the step of removing the sock comprises contacting the sock with a solvent capable of dissolving the sock.

7. The method of claim 1, wherein the step of removing the sock from the silicone elastomer comprises contacting the sock with an organic solvent selected from methylene chloride, chloroform, acetone, tetrahydrofuran and combinations thereof.

8. The method of claim 2, wherein the sock comprises a mesh including a plurality of eyes having an average size between about 100 μm×100 μm to about 2000 μm×2000 μm.

9. A component of a breast implant shell made by the method of claim 1.

10. A breast implant, comprising:
a silicone shell having an inner surface, defining a cavity configured to be filled with a filler material, and a textured outer surface, the shell made by the steps of
providing a mandrel;
applying a polymeric mesh sock to the mandrel;
contacting the sock with a silicone dispersion;
at least partially curing the silicone dispersion to form a silicone elastomer with the sock in contact therewith; and
after the silicone is at least partially cured, removing the sock from the silicone elastomer to obtain a textured silicone material;
forming the textured silicone material into a shell of a breast implant.

11. The implant of claim 10 wherein the steps further comprise applying at least one additional polymeric mesh sock onto the mandrel prior to the step of at least partially curing.

12. The implant of claim 10 wherein the step of contacting the sock with the silicone dispersion is performed after the step of applying the sock.

13. The implant of claim 10, wherein the sock is comprised of at least one of Vicryl 910, poly (L-lactic acid-co-trimethylcarbonate), polycaprolactone, poly(L-lactic acid), poly(methyl methacrylate) and poly(lactic-co-glycolic acid).

14. The implant of claim 10 wherein the sock is comprised of poly(L-lactic acid.

15. The implant of claim 10, wherein the step of removing the sock comprises applying to the sock a solvent capable of dissolving the sock.

16. The implant of claim 10, wherein the step of removing the sock comprises contacting the sock with an organic solvent selected from methylene chloride, chloroform, acetone, tetrahydrofuran and combinations thereof.

17. The implant of claim 10, wherein the sock comprises a mesh including a plurality of eyes having an average size between about 100 μm×100 μm to about 2000 μm×2000 μm.
